(19) United States
(12) Patent Application Publication
(10) Pub. No.: US 2017/0296228 A1
(43) Pub. Date: Oct. 19, 2017

(54) Trocar System

(71) Applicants: Siegfried Riek, Rottweil (DE); Karl-Heinz Bachmann, Villingendorf (DE); Thomas Gaiselmann, Villingendorf (DE)

(72) Inventors: Siegfried Riek, Rottweil (DE); Karl-Heinz Bachmann, Villingendorf (DE); Thomas Gaiselmann, Villingendorf (DE)

(21) Appl. No.: 15/639,533

(22) Filed: Jun. 30, 2017

Related U.S. Application Data
(62) Division of application No. 14/129,886, filed on Dec. 27, 2013.

(30) Foreign Application Priority Data
Jun. 30, 2011 (DE) 10 2011 107 615.1

(51) Int. Cl. A61B 17/34; A61B 1/07; A61B 17/32; A61B 1/05; A61B 17/00

(52) U.S. Cl. CPC A61B 17/3423; A61B 17/3496; A61B 1/07; A61B 17/32002; A61B 17/3474; A61B 1/05; A61B 17/3478; A61B 2017/00862; A61B 2017/00867; A61B 2017/320024; A61B 2017/3456

(57) ABSTRACT

A trocar system, having a trocar, a trocar sleeve, an optical channel extending coaxially in the trocar for receiving an optical unit, and a hollow transparent distal tip of the trocar, which can be observed from the interior by means of the optical unit, wherein a working channel extends continuously from the proximal end to the distal end in the trocar and opens into an outlet opening in the region of the distal end, and wherein the inner wall of the working channel is formed at least in an axially continuous sub-region of the circumference thereof by the trocar sleeve.

### CROSS REFERENCE TO RELATED APPLICATIONS

This application is a divisional application of and claims the benefit of U.S. application Ser. No. 14/129,886, filed Dec. 27, 2013, which is a §371 National Phase of PCT/EP2012/002479, filed Jun. 12, 2012, which claims priority to German Patent Application No. 10 2011 107 615.1, filed Jun. 30, 2011, the entireties of which are incorporated by reference herein.

### BACKGROUND

The application relates to a trocar system according to the preamble of claim 1.

Trocar systems that are intended for use in minimally invasive surgical applications typically consist of a trocar that is used to create an opening in a body cavity (for example, the abdomen) and a trocar sleeve that is placed and remains inside said opening constituting an access point to the inside of the body cavity for the surgical procedure. The trocar includes a distal tip for penetrating the body tissues, for example the abdominal wall, and serves to create an opening. The tip of the trocar can be configured as pointed, cutting or dull. A pointed tip, for example, has the shape of a three-edged pyramid. Cutting tips include a blade for a tissue incision that is subsequently dilated by a cone-shaped tip. Dull tips are distally rounded, which means that very high penetration pressures must be applied to them if they are used for opening up tissue layers. Correspondingly, dull tips are essentially only used to dilate a previously created lesion.

These trocars, particularly pointed and cutting trocars, are associated with risks; upon penetrating the abdominal wall, they may cause injury to internal organs that can adhere to the peritoneum due to internal adhesions, such as, for example, the bowel and/or blood vessels in the abdominal wall or retroperitoneum. To reduce this risk, so-called optical trocars are in use, for example, as disclosed in U.S. Pat. No. 5,685,820 A. The distal tip on these optical trocars is configured as a hollow, transparent cone that can be observed from the inside through an optical unit, which is taken up inside an optical channel extending coaxially inside the trocar. With the transparent tip, the optical trocar gives access to a three-dimensional view of the tissue layers of the abdominal wall through which the trocar passes. This affords the surgeon with a sensed idea for the motion, speed and position of the trocar tip during penetration. In particular, it is possible to detect any adhesions that may be present between the bowel and the peritoneum at the insertion point prior to penetrating the peritoneum. Nevertheless, the high penetration pressures needed for passing through the fascia and the peritoneum still remain problematic. Although, conceivably, it is possible to reduce the necessary penetration pressures by the use of cutting blades that are disposed on the trocar tip, the use of a blade poses new injury risks for the bowel during the penetration step. To reduce the penetration pressure, and as a compromise between reducing pressure and injury risk, the known optical trocar is used with or without scraping runners, wherein, however, even in this case, the penetration pressures are still relatively high, and permanently rotating trocar motions are required. Moreover, there results the so-called tenting effect, whereby the trocar presses the tissue layers that require high penetration pressures in a tent-like fashion into the abdomen, possibly advancing them into close proximity of the retroperitoneum. When these layers are opened, they give way to the penetration pressure suddenly, and the tip penetrates the abdomen all of a sudden, possibly making it difficult for the surgeon to control the sudden trocar motion in an effort to avoid injuring internal organs or large vessels in the retroperitoneum with the tip of the trocar. To avoid this problem, many surgeons work with a mini-laparotomy. This procedure envisions the placement of a skin incision according to the classical technique through the abdominal wall, the abdomen is opened and a trocar sleeve then inserted into the opened peritoneum.

Sealing a pneumoperitoneum is problematic herein, because the opening in the abdominal wall, which is created in this way, is larger than the opening that would have been created with a trocar-driven perforation. The open incision of the abdominal wall is in contradiction, however, to the stated goal of a minimally invasive surgical technique.

Therefore, it is the object to provide a trocar that utilizes all of the advantages of the optical trocar without requiring, however, high pressures for the tissue penetration.

This object is achieved by a trocar system having the features and structures disclosed herein.

Advantageous embodiments are indicated in the dependent claims.

The disclosure provides for using the trocar not only as a passive tool that is manually guided by an axial force and, if necessary, a rotational movement through the tissue layers. Rather, the trocar can be associated with various active surgical instruments that are inserted through at least one working channel and can be extended at the distal tip of the trocar. This allows the surgeon to engage in surgical work directly in the surgical field at the distal tip of the trocar, without the need of having to create a further access point in addition to the trocar. Taking advantage of the optical unit and the transparent distal tip of the trocar, the surgeon has visual contact while executing the surgical procedures by means of the instruments that are extended through the working channels. A large number of different instruments is available in miniaturized design configurations and suitable for traversing the working channels. A corresponding multitude of different surgeries is thus made possible with the trocar system.

For the insertion of the trocar through the different tissue layers, particularly the abdominal wall, it is possible to provide a miniaturized pair of scissors or a blade, which are extended through the distal trocar tip, to thereby separate or cut into the respective tissue layer in front of the distal tip. Particularly the resilient tissue layers, for example, of the fascia and the peritoneum can be opened in this manner while maintaining visual contact; a small incision is placed, followed by the subsequent penetration of the tip of the trocar into this incision without applying any major pressure, particularly avoiding the tenting effect, then dilating the incision and penetrating the tissue layer. Similarly to the open laparotomy technique, in this way, it is possible to visualize and prepare tissue layers that rest against the distal tip of the trocar to allow for an almost pressureless, and thereby risk-free, penetration of the tissue layers. The semi-transparency of the peritoneum therein allows for detecting adhesions by means of the trocar prior to opening the tissue layers. This is not possible with an open la [symbol] parotomy.

Furthermore, it is possible to guide pairs of tweezers or forceps through the working channels to hold the tissue at the distal trocar tip, which can be advantageous particularly when an incision is placed by means of a pair of scissors or a blade inserted through a further working channel.

Moreover, with the first perforation it is, furthermore, advantageously possible to insert a miniaturized Veress needle through the working channel. Using this Veress needle, it is possible to perforate the peritoneum under visual contact in order to then insufflate the abdominal cavity by means of the Veress needle.

Moreover, it is also possible to guide clamps or coagulation instruments through the working channels, and extending the same through the distal tip. Using these instruments, it is possible to clamp and/or coagulate such vessels.

Moreover, it is also possible to extend miniaturized morcellators through the distal tip.

Moreover, it is also possible to insert a miniaturized digital camera through a working channel, for example, for the purpose of documenting the surgery. It is, furthermore, possible to insert fiber-optic light guides, illumination systems or optical means through the working channel.

Therefore, the trocar system thus allows for penetrating, in particular, the abdominal wall, by inserting the trocar, for example, until the distal tip of the trocar reaches the fascia. A clamp is then extended through one working channel that holds the fascia, while a pair of scissors is extended through another working channel that is used to open the fascia. This step is achieved while the surgeon has visual contact through the transparent tip of the trocar. The tip of the trocar is now inserted in the thus created opening in the fascia, and wherein the further dila [symbol] ftation occurs without tissue trauma and with minimal penetration pressure. When the tip of the trocar reaches the peritoneum and no adhesions are found, the peritoneum can be opened correspondingly by the use of scissors and, if necessary, a clamp. No remarkable penetration pressure is needed, whereby the tenting effect and any of the related associated risks are avoided. This process is analogous to the usual preparative steps in the context of an open mini-laparotomy. However, in contrast to this known preparative procedure, no larger incision is necessary than the size cut that is needed for accommodating the insertion of the trocar to be able to prepare the tissue layers under visual contact. Alternately, once the peritoneum has been reached, it is possible to extend a Veress needle through a working channel by which the peritoneum is then penetrated under visual contact to then insufflate the abdomen with carbon dioxide (CO2). As soon as, due to the insufflation, the pe [symbol] ritoneum has been separated from the bowel, the Veress needle is retracted, and the tip of the trocar is inserted through the opening that has been created in this manner in order to dilate said opening without causing tissue trauma and with the application of minimal pressure.

With the trocar system, it is possible to place a trocar sleeve, which then serves as an access channel for the subsequent minimally invasive surgery. In the same manner, it is possible to work surgically through a single port using the trocar system. In this instance, the trocar remains, along with the optical unit in the trocar sleeve, and the working channels serve as the only access point for the subsequent minimally invasive surgery, wherein the surgical instruments are inserted through the working channels. The surgery is conducted under visual contact with the surrounding area through the distal tip of the trocar. Alternately, after the placement of the trocar sleeve, it is possible to replace the trocar with an optical unit to provide an open view of the surgical field.

The instruments that can be used in connection with the trocar system are essentially miniaturized surgical instruments that are known from the prior art. They include an extendable working element at the distal tip of the trocar, while, on the end that remains proximally outside of the working channel, the proximal actuating elements of the miniature instruments are disposed. The instruments can be configured therein with a rigid, flexible or semiflexible shaft. Semi-flexible instruments can be elastically preloaded in such a manner that the distal working elements thereof bend relative to the center axis of the trocar upon exiting from the distal tip of the trocar to allow for engaging in preparative work directly in front of the transparent tip.

Alternately, it is also possible to dispose a small guide tube, axially displaceable and rotatable, inside the working channel, through which the miniature instrument is traversed. In the distal end region thereof, the guide tube is elastically preloaded to bend. The guide tube is preferably made of a memory alloy with super-elastic properties, for example of nitinol. When the small guide tube is distally pushed out of the working channel, the distal end thereof curves away from the longitudinal axis that is defined by the working channel, wherein the angle of deflection relative to the longitudinal axis increases the farther the distal end of the small guide tube exits from the working channel. By rotating the small guide tube inside the working channel, it is possible to rotate the direction of the deflection around the longitudinal axis. By axially displacing and rotating the small guide tube, it is thus possible to exercise a three-dimensional control over the direction of exit and the positioning of the distal working element of the miniature instrument. Adjusting means, that are provided at the proximal end, allow for the axial and rotational movement of the small guide tube inside the working channel.

To prevent gas from escaping from the insufflated abdominal area, a valve can be envisioned to provide a proximal seal for the working channels, when no instrument is present inside the working channel. A valve of this kind can be formed, in particular, by a sealing lip, which is known from the prior art, that permits an instrument to pass through it and then seals such an inserted instrument along the external circumference thereof.

Furthermore, unused working channels can be sealed off by a mandrin that closes off the distal outlet opening of the working channel to prevent contaminants from entering the working channel.

The working channels in the trocar system that extend from proximal to distal can be produced in different manners. The different embodiments share the characteristic that the trocar sleeve forms the interior wall of the working channels at least in a partial area of the cross-section of the working channels.

In one embodiment, the at least one working channel is formed as an axially continuous bore inside the wall of the trocar sleeve. The material of the trocar sleeve thus encloses the working channel on the entire circumference thereof. This design is more complex in terms of manufacturing; however, it has the advantage that the working channels in the trocar sleeve, which are enclosed along the total circumference thereof, are available even after the trocar has been extracted following the placement of the trocar sleeve. The working channels of the trocar sleeve can then be used for the surgery in addition to the internal lumen of the trocar sleeve. This can be especially advantageous for the single-port surgical technique.

In terms of manufacturing, it is easier if at least one working channel is formed between the exterior jacket surface of the trocar and the interior wall surface of the trocar sleeve.

The at least one working channel therein can be configured as an axially continuous groove in the interior wall surface of the trocar sleeve, which is closed off by the exterior jacket surface of the trocar. The axially continuous groove can be easily produced. The wall strength of the trocar sleeve can be smaller than in the first configuration. It is further advantageous when it is possible to use any kind of trocar.

Alternately, the at least one working channel can be formed by an axially continuous groove in the exterior jacket surface of the trocar, which is then closed off by the interior wall surface of the trocar sleeve. In this configuration, the trocar is embodied, while it is possible to use a conventional trocar sleeve. It is further advantageous that the smallest wall strength of the trocar sleeve is required herein.

In another configuration, the at least one working channel can be formed by axially continuous grooves in the exterior jacket surface of the trocar and in the interior wall surface of the trocar sleeve. When they are in their angle at circumference position, the grooves are congruent relative to each other, whereby the groove in the trocar and the groove in the trocar sleeve each constitute half of the internal cross-section of the working channel.

Moreover, it is possible to form the at least one working channel by a small tube that is attached to the exterior on the jacket surface of the trocar sleeve.

Finally, a configuration, where an annular-like gap remains between the exterior jacket surface of the trocar and the interior wall surface of the trocar sleeve, is possible as well; and at least one shell is inserted in said annular-like gap that leaves free an angle at circumference area that extends in an axially continuous fashion, whereby the working channel is formed.

The working channels generally extend axially parallel in relation to the trocar sleeve or offset relative to the instrument axis, or also helically about the instrument axis. However, in the proximal inlet area and/or in the distal outlet area, it is possible for the working channels to be outwardly offset relative to the axially parallel direction.

An axially continuous bore is preferably disposed inside the wall of the trocar sleeve that serves as an insufflation channel. This offers the possibility of easy insufflation even during surgery, which is advantageous, particularly in the context of the single-port surgical technique. It is understood that the insufflation can also be effected through the internal lumen of the trocar sleeve, as is customary with known trocar systems.

A valve housing can be mounted on the trocar sleeve in a manner that is known from the prior art, and which provides a proximal closure for the trocar sleeve, when no trocar, nor an instrument is present inside the trocar sleeve. When the trocar or a surgical instrument is inserted through the trocar sleeve, they are sealed along the circumference thereof to prevent the insufflation gas from escaping. The proximal inlet ends of the working channels are disposed outside of the valve housing, allowing for the possibility of traversing the respective instruments through the working channels independently of the valve housing.

The features and structures of the present disclosure will be explained in further detail below based on the embodiments as illustrated in the drawings. Shown are as follows:

### BRIEF DESCRIPTION OF THE DRAWINGS

FIG. 1 is a representation of an axial section through the trocar system in a first configuration;

FIG. 2 is a representation of a cross-section of the trocar system according to the sectional line A-A from FIG. 1;

FIG. 3 is a representation of a corresponding cross-section of the trocar system by way of a second configuration;

FIG. 4 is a representation of a corresponding cross-section of a trocar system by way of a third configuration;

FIG. 5 is a representation of a corresponding cross-section of the trocar system by way of a fourth configuration;

FIG. 6 is a corresponding representation of the trocar system by way of a fifth configuration; and

FIG. 7 is a corresponding cross-section of the trocar system by way of a sixth configuration.

### DETAILED DESCRIPTION OF THE ILLUSTRATED EMBODIMENTS

Only those parts of the trocar system are represented in the drawings and in the following description that are embodied. Otherwise, the trocar system is compliant with the known prior art, wherein all of the variations that are known from the prior art presently fall under the scope of protection.

The trocar system includes a trocar 10. The trocar 10 takes the shape of a rigid, oblong, cylindrical tube that is manufactured of metal or plastic. The internal lumen of the trocar 10 constitutes an optical channel 12 that extends coaxially from the proximal end to the distal end. The distal tip 14 of the trocar 10 has, for example, the shape of a cone with a rounded, blunt tip. The jacket of the cone can be convexly arched, if necessary. The cone-shaped tip 14 is hollow on the inside and made of a thin-walled, transparent material, particularly a transparent plastic. An optical unit can be inserted into the optical channel 12 of the trocar 10, which is constituted, for example, as a rod-lens optics means or a camera chip. and an illumination system can be integrated therein. When the used optical unit is inserted, the distal end thereof is approximately in the region of the base area of the cone-shaped tip 14. With the optical unit, it is possible to illuminate and observe the distal tip 14 from the inside. This way, it is possible to observe the tissue that rests against the exterior side of the dista-1 tip 14.

A trocar sleeve 16 can be pushed over the trocar 10. At the proximal end of the trocar sleeve 16, a valve is disposed in a manner that is known from the prior art, which is why no further explanation is needed here, and that seals the trocar sleeve 16, when no instrument is present in the trocar sleeve 16. If the trocar 10 or a further instrument is inserted through the trocar sleeve, the valve seals such a trocar or instrument along the circumference thereof.

An insufflation channel 18 can be configured inside the trocar sleeve 16. The insufflation channel 18 extends as an axially parallel bore inside the wall of the trocar sleeve 16. A connection point 20 for the insufflation gas is disposed at the proximal end of the insufflation channel 18. At the distal end, the insufflation channel 18 opens at the open distal end of the trocar sleeve 16. Alternately, the connection point 20 can also lead into the internal lumen of the trocar sleeve 16, whereby the internal lumen constitutes the insufflation channel.

When the trocar sleeve 16 is pushed onto the trocar 10, the distal end of the trocar sleeve 16 follows at the proximal end of the tip 14 of the trocar 10, whereby the exterior contour of the tip 14 transitions in a stepless manner into the exterior contour of the trocar sleeve 16.

The trocar system includes at least one working channel 22 that extends inside the trocar sleeve 16 from proximal to distal. The working channel 22 generally extends in an axially parallel fashion relative to the center axis of the trocar sleeve 16. At the proximal end, the working channel 22 includes an inlet opening 24, which is disposed outside of the valve, whereby said inlet opening is freely accessible. The inlet opening 24 can be disposed as offset relative to the axially parallel direction. At the distal end, the working channel 22 opens into a free outlet opening 26 in the area of the distal end of the trocar sleeve 16. The axis direction of the outlet opening 26 can also be disposed as offset relative to the axially parallel direction. In the embodiments as presently shown, two working channels 22 are disposed diametrically relative to each other, respectively. However, it is also possible to provide only a single working channel 22 or even more than two working channels 22. If two or more working channels 22 are provided, they are preferably disposed at the same angular distances relative to each other.

The working channels 22 serve for inserting miniature instruments, which are known from the prior art. These miniature instruments are any instruments that are appropriate for the respective application scenario, as known from the prior art. The miniature instruments include an oblong, rigid, flexible or semi-flexible shaft, and at the distal end of which a working element is disposed, respectively, which can be actuated by means of an actuating element that is disposed at the proximal end of the shaft. The miniature instrument is inserted into the working channel 22 advancing from the proximal end, and wherein it is possible for the instrument to be sealed at the proximal inlet opening 24. An inlet opening 24 that is disposed in an outwardly offset position facilitates the insertion of the miniature instrument laterally in front of the valve of the trocar sleeve 16. The miniature instrument is advanced inside the working channel 22 until the distal working element is extended through the outlet opening 26 and is able to become engaged in the surgical field in front of the distal tip 14. If the trocar sleeve 16 and the working channels 22 are manufactured from a transparent plastic material, the observer is able to see the inserting and advancing operation of the miniature instrument from the outside.

If necessary, a mandrin, presently not shown, can be inserted in any unused working channel 22 closing the outlet opening 26 in a flush design, thereby preventing contaminants from penetrating the unused working channel 22.

The at least one working channel 22 can be obtained in different ways.

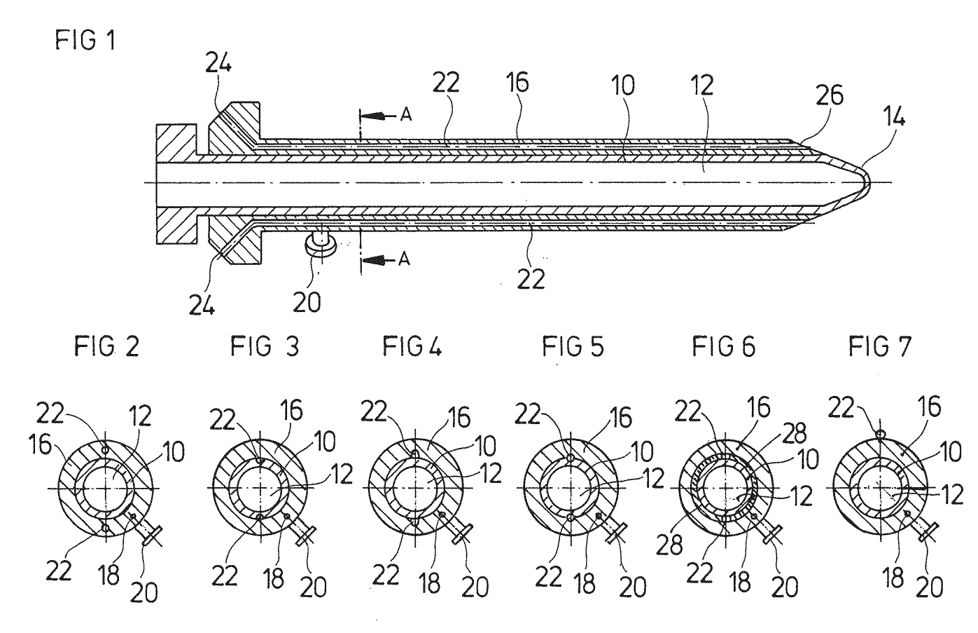

In a configuration as embodied in FIGS. 1 and 2, the at least one working channel 22 is formed as an axially continuous bore inside the solid wall of the trocar sleeve 16. The material of the trocar sleeve wall thus encloses the entire cross-sectional circumference of the working channel 22. This configuration is particularly suited, when the trocar 10 is replaced with an optical unit after the trocar sleeve 16 has been properly positioned.

In a second embodiment, as shown in FIG. 3, the at least one working channel 22 is formed by a groove that extends axially in the exterior jacket surface of the trocar 10. The circumferential area of the cross-section, that is open on the exterior side of the trocar 10, is closed off by the interior wall surface of the trocar sleeve 16, which is pushed thereon to form the working channel 22 that is closed along the circumference.

In a third embodiment, as shown in FIG. 4, the at least one working channel 22 is formed by an axially continuous groove formed inside the interior wall surface of the trocar sleeve 16. The open area of the groove on the interior side of the trocar sleeve 16 is closed off by the exterior jacket surface of the trocar 10 in order to form the working channel 22 that is closed along the entire cross-sectional circumference thereof.

In the fourth embodiment, as shown in FIG. 5, the exterior jacket surface of the trocar 10 as well as the interior wall surface of the trocar sleeve 16 are formed each as axially extending grooves, which are congruent in terms of the angular position relative to the trocar axis. The two grooves, which become congruent, supplement each other to form the cross-section of the at least one working channel 22.

In a fifth embodiment, as shown in FIG. 6, the exterior diameter of the trocar 10 is somewhat smaller than the interior diameter of the trocar sleeve 16, whereby an annular-like gap remains free between the interior wall surface of the trocar sleeve 16 and the exterior jacket surface of the trocar 10. A number of circular-cylindrical shells 28 are inserted in this gap that corresponds to the number of working channels 22. In the circumferential direction, an angle at circumference area remains free between the shells 28, whereby the axially continuous working channels 22 are formed. On the radial exterior side thereof, the working channel 22 is constituted by the interior wall surface of the trocar sleeve 16; on the radial interior side thereof, it is formed by the exterior circumferential surface of the trocar 10 and, at both circumferential sides surfaces thereof, said channel is constituted of the shells 28.

In a sixth embodiment, as shown in FIG. 7, the at least one working channel 22 is formed by a small tube that is mounted on the outside to the exterior jacket surface of the trocar sleeve 16 (for example, by gluing, point-welding or as cast into the jacket surface.

### LIST OF REFERENCE SIGNS

10 Trocar
12 Optical channel
14 Tip
16 Trocar sleeve
18 Insufflation channel
20 Connection
22 Working channel
24 Inlet opening
26 Outlet opening
28 Shells

1-13. (canceled)

14. A trocar system, comprising:
a trocar with a hollow, transparent distal tip;
a trocar sleeve;
an optical channel extending coaxially inside the trocar for receiving an optical unit, wherein the transparent distal tip can be observed from inside the trocar by the optical unit,
a working channel that continuously extends from a proximal end to a distal end of the trocar sleeve and opens in an area of the distal end into an outlet opening,
wherein an interior wall of the working channel is formed by the trocar sleeve at least in one axial partial area of a circumference thereof;
wherein the working channel is a continuous bore inside a wall of the trocar sleeve, wherein the trocar sleeve forms the interior wall of the working channel around an entire circumference thereof.

15. The trocar system according to claim 14,
wherein an axially continuous bore is located inside the wall of the trocar sleeve as an insufflation channel for introducing a gas for insufflation.

16. The trocar system according to claim 14,
wherein a valve is disposed proximally on the trocar sleeve that allows for a sealed passage of the trocar and that closes the trocar sleeve off when the trocar is not inserted, and wherein the proximal inlet opening of the working channel is disposed outside of the valve.

17. The trocar system according to claim 14,
wherein a miniature instrument can be inserted through the working channel, such that a distal working element of the miniature instrument exits at the distal end from the outlet opening of the working channel, and wherein a proximal actuating element of the miniature instrument is disposed proximally outside of an inlet opening of the working channel.

18. The trocar system according to claim 17,
wherein the miniature instrument is a pair of scissors, a blade, a pair of tweezers, a clamp, a coagulation instrument, a Veress needle, a digital camera, a fiber-optic light guide, an illumination system or an optical unit.

19. The trocar system according to claim 18,
wherein in the working channel, a small guide tube is disposed in an axially displaceable and rotatable manner, through which the miniature instrument can be traversed, and wherein the small guide tube is elastically preloaded to curve at least in the area of the distal end thereof.

20. The trocar system according to claim 19,
wherein the small guide tube is made of a memory material having super-elastic properties.

21. A trocar system, comprising:
a trocar, the trocar comprising:
a trocar jacket surface defining an exterior circumferential surface of the trocar;
an optical channel extending inside the trocar for receiving an optical unit;
a transparent distal tip of the trocar, wherein an area exterior of the distal tip can be observed via the optical channel;
a trocar sleeve that has a trocar sleeve internal surface covering an elongated portion of the trocar jacket surface of the trocar; and
a working channel comprising a continuous bore inside a wall of the trocar sleeve, wherein the trocar sleeve forms an interior wall of the working channel around an entire circumference thereof.

22. The trocar system according to claim 21,
wherein an axially continuous bore is located inside the wall of the trocar sleeve as an insufflation channel for introducing a gas for insufflation.

23. The trocar system according to claim 21,
wherein a valve is disposed proximally on the trocar sleeve that allows for a sealed passage of the trocar and that closes the trocar sleeve off when the trocar is not inserted, and wherein the proximal inlet opening of the working channel is disposed outside of the valve.

24. The trocar system according to claim 21, wherein a miniature instrument can be inserted through the working channel, such that a distal working element of the miniature instrument exits at a distal end from an outlet opening of the working channel, and wherein a proximal actuating element of the miniature instrument is disposed proximally outside of an inlet opening of the working channel.
